(19) United States
(12) Patent Application Publication
(10) Pub. No.: US 2006/0229284 A1
(43) Pub. Date: Oct. 12, 2006

(54) Enhanced occlusive effect photodynamic therapy

(76) Inventors: Paula Ann Mahoney, Woodinville, WA (US); Louise Ellen Culham, Auburn, WA (US); Brian William McIlroy, Sammamish, WA (US); Gregory Lee Heacock, Auburn, WA (US); Andrew Michael Peter Hamilton, Pinner (GB); John Marshall, Farnborough (GB)

Correspondence Address: MCANDREWS HELD & MALLOY, LTD, 500 WEST MADISON STREET, SUITE 3400, CHICAGO, IL, 60661, US

(21) Appl. No.: 11/301,743

(22) Filed: Dec. 13, 2005

Related U.S. Application Data
(60) Provisional application No. 60/636,852, filed on Dec. 15, 2004.

(51) Int. Cl. A61K 31/409; A61K 31/555; A61H 33/00

(52) U.S. Cl. 514/185; 514/410; 607/86

(57) ABSTRACT

This invention discloses methods of treating neovasculature diseases of the eye through the administration of a photosensitizing agent and subsequent exposure to light of specific wavelength sufficient to photoactivate the photosensitizing agent to occlude one or more vessels in the neovasculature for an extended period of time. Diseases treatable under this invention, include, for example: diabetic retinopathy; macular degeneration; subfoveal choroidal neovascularization, malignant uveal melanomas and other maladies of the human or animal eye or body.

### RELATED APPLICATIONS

This application claims the benefit of currently pending U.S. Provisional Patent Application No. 60/636,852 filed on Dec. 15, 2004.

All patent applications noted above are incorporated by reference in their entirety to provide for continuity of disclosure.

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

1. Field of the Invention

This invention relates generally to the field of medicine and pharmacotherapeutics with photosensitizing agents or other energy activated agents. Specifically, this invention relates to methods useful for the treatment of neovascular diseases of the eye. The invention involves the delivery of a photosensitizing agent that is activated by light to produce enhanced vessel occlusion within neovascular tissue for an extended period of time.

2. Description of the Related Art

Neovascular diseases of the eye include, for example, diabetic retinopathy, age-related macular degeneration and neovasculature growth induced by angiogenic factors or resulting from tumor cells, themselves. Diabetic retinopathy is characterized by a number and variety of microvascular changes which can result ultimately in adverse visual changes and vision loss. In many cases the microvascular changes are due to or associated with upregulation of angiogenesis receptors and factors of ligands which lead to new vessel formation, changes in vascular permeability, and possibly other alterations in vessel morphology. These changes may lead to hemorrhage, edema, ischemia, and other problems resulting in vision dysfunction (see: Aiello et al., Diabetes Care, 21:143-156, 1998).

Treatments for the various forms of, and problems associated with, diabetic retinopathy, for example, include laser photocoagulation, vitrectomy, cryotherapy, and membranotomy. All of these clinical therapies and procedures are associated with problems and side effects. For example, the side effects and complications related to panretinal laser photocoagulation, the most common present treatment for diabetic retinopathy, include: decreased visual acuity, increased macular edema, transient pain, exudative retinal detachment, and inadvertent foveolar burns.

Further, age-related macular degeneration (“AMD”) is the leading cause of blindness in the United States among individuals 65 or older. One form of AMD is characterized by formation of choroidal neovessels which can lead to a number of pathologic conditions resulting in visual dysfunction and loss. As with diabetic retinopathy, angiogenesis plays a key role in the formation of these neovessels. The proliferation and/or leakage of choroidal neovessels associated with AMD can contribute to irreversible damage of photoreceptors. Thus, current treatment of AMD, like that of diabetic retinopathy, involves the use of laser photocoagulation. However, because photocoagulation relies upon the gross thermal destruction of the choroidal neovascular tissue, damage to the retina and surrounding choroidal tissue often results, leaking of neovasculature, and the like. Furthermore, recurrences of such tissue growth or leakage of such tissue after photocoagulation therapy are common. (see: Schmidt-Erfurth et al., Greafe's Arch Clin Exp Opthamol, 236:365-374, 1998).

In photodynamic therapy (PDT), classes of photoreactive compounds, also known as “photosensitizers”, are excited with specific illumination wavelengths in order to treat diseased or undesirable tissue. In general, PDT treatment utilizing light is a two-step treatment process. Such treatment is generally performed by first administering a photosensitive compound systemically or topically, followed by illumination of the treatment site at a wavelength or waveband of light from a laser which closely matches the absorption spectra of the photosensitizer. In doing so, singlet oxygen and other reactive species are generated leading to a number of biological effects resulting in damage to the endothelial membranes and ultimately to clotting or occlusion of the neovasculature.

Further, photosensitizers suitable for PDT may be activated by at least one wavelength of light (“the excitation wavelength”) and are used in combination with light sources of appropriate excitation wavelength, often provided as laser light, to treat targeted tissue in a variety of eye, cardiac, oncological and other disease conditions. Additionally, light sources for PDT, are generally high powered lasers are usually employed in order to shorten the procedure time (see: Strong et al., U.S. Pat. Nos. 5,756,541 and 5,910,510; and Mori et al., U.S. Pat. No. 5,633,275; see more generally, W. G. Fisher, et al., Photochemistry and Photobiology, 66(2):141-155, 1997).

Thus, the two important and related components of a photo reactive treatment system are the photosensitizer and the excitation light source and apparatus for supplying the light appropriately to targeted tissue. Accordingly, much research is being directed into both of these areas. With regard to apparatus, conventional approaches to PDT are challenged by requirements of light exposure of desired intensities, duration, shape, and timing when photosensitizers are present in the diseased tissue. Inappropriate illumination, such as misdirected or misshaped illumination, or excessive intensity, could cause photosensitizers to unnecessarily injure normal healthy tissue. In the case of the photosensitizer, it must be non-toxic, a non-irritant or at least well tolerated, and when activated its vessel-closure (“occlusion”) effects should be effective with minimal delay.

By way of example and to further illustrate, in (wet) age-related macular degeneration (AMD), glaucoma, and diabetic retinopathy (DR), photosensitizers activated by light via PDT treatment may be used to inhibit or retard disease progression, as commonly indicated by abnormal new vessel growth (known as “neovascularization”), within diseased eye tissue and to reduce or eliminate any potential factors associated with leaking new vessels.

While it is known in the prior art to treat neovasculature with PDT procedures, using laser or other light of appropriate wavelength, these procedures have not entirely stopped the re-growth of abnormal new vessels and/or re-opening of previously closed abnormal new vessels. For example, as an alternative to photocoagulation, photodynamic therapy has been proposed as a means of treating AMD (see: Strong et al., “Vision through photodynamic therapy of the eye,” U.S. Pat. Nos. 5,756,541 and 5,910,510; and Mori et al., “Photochemotherapeutical obstruction of newly-formed blood vessels,” U.S. Pat. No. 5,633,275). Although this form and example of PDT represents an improvement over photocoagulation, clinical experience has established that the therapy must be repeated on a regular basis, typically every 3 months due to re-growth or re-opening of the vessels of the neovasculature (see: Schmidt-Erfurth et al.).

Moreover, based on published data of commercialized ocular PDT, it is has been observed as being medically necessary and/or desirable to follow up and re-treat subject eyes at regular intervals to effect closure of any newly generated vessels of the neovasculature being treated, and to re-close previously treated vessels that have re-opened. Such negative outcomes can be further observed in the published data relating to use of verteporfin, also known as Visudyne® [a trademark of QLT of Vancouver, Canada], utilized as a photosensitizer in PDT procedures to treat age-related macular disease (AMD). See, e.g.,: Visudyne® package insert; [AA] “Photodynamic Therapy with Verteporfin for Choroidal Neovascularization Caused by Age-related Macular Degeneration: Results of a Single Treatment in a Phase 1 and 2 Study”, Joan W Miller et al., ARCH OPHTHALMOL, vol. 117 September 1999; [AB] “Photodynamic Therapy with Verteporfin for Choroidal Neovascularization Caused by Age-related Macular Degeneration: Results of Retreatments in a Phase 1 and 2 Study”, Schmidt-Erfurth et al. ARCH OPHTHALMOL, vol. 117 September 1999 [AC] “Short-term Reaction of Choroidal Neovascularization and Choriocapillaris to Photodynamic Therapy in Age-related Macular Degeneration”, Eter et al. European Journal of Ophthalmology, 7 vol. 13 pp 687-692 (2003). Each of these studies ([AA], [AB], and [AC]) is discussed further below.

Study [AA] related to ocular PDT treatment with verteporfin as a photosensitizer on 128 subjects with subfoveal choroidal neovascularization (CNV). The study indicated that after about 4 to about 12 weeks following PDT treatment, fluorescein leakage reappeared in almost all cases. Further, progression of classic CNV beyond the area of CNV identified before treatment was noted in 51% of the cases that were followed for 3 months after a single PDT treatment. As a result, the study concluded that PDT treatment with verteporfin achieved “short term” cessation of fluorescein leakage from CNV without loss of vision or growth of classic CNV in some patients with AMD.

Study [AB] was a follow-up study regarding 31 subjects who had been re-treated with verteporfin PDT treatment. The study indicated that follow-up examinations occurred within 16 to 20 weeks after initial treatment. The study also indicated that in most cases fluorescein leakage reappeared within 4 to 12 weeks after re-treatment. However, compared to baseline leakage, the leakage activity appeared to be reduced. Yet, this particular study concluded that repetitive verteporfin PDT treatment can achieve only “short-term” cessation of leakage without loss of visual acuity. Moreover, this study suggests that re-treatments “may achieve progressive cessation of leakage”, prevent further growth of CNV and subsequent visual loss, but cautions: “persistent absence of leakage was not achieved at some point between weeks 4 and 12 even at the highest light dose.”

Study [AC] was designed to determine the number of primary angiographic non-responders to verteporfin PDT treatment, and to determine the rate of re-perfusion of CNV after 5 weeks by testing 36 eyes according to the TAP regimen. In general, a TAP regimen involves selection of patients that are over 50 years of age, have been diagnosed with AMD, have had an examination within 1 month onset of visual symptoms associated with AMD, and had confirmation of CNV via an ICG (indocyanine green angiography) or FA (fluorescein angiography) procedure, with the FA procedure being preferred.

Examination of the subjects at 1 and 5 weeks was carried out using both fluorescein and indocyanine green angiography. Before treatment, all eyes (36) showed leakage; after 1 week, 83% of the subject eyes maintained CNV closure; and after 5 weeks only 9% showed closure, with 91% (excluding one eye removed from test data) showing leakage had recommenced.

Additionally, referring to data from other TAP reports of this study, subjects had follow-up examinations 3 months after initial PDT treatment. Those follow-up examination reports indicated: “At that time, 92.8% of eyes with classic CNV present displayed leakage from classic CNV again and were scheduled for retreatment (90.8%).” Thus, this [AC] study confirms the TAP data in finding that in as short a period of time as 5 weeks (following a first verteporfin PDT treatment) leakage from CNV recurred in a comparable number of cases, namely 91%.

However, the present art lacks an effective method of treating neovasculature diseases, in particular neovasculature disease of the eye, using a PDT methodology, which reduces or prevents leaking, re-leaking, and/or re-opening of one or more vessels (e.g., blood vessels) in previously treated neovascular tissue or newly grown, developed, or recurrent neovascular tissue for an extended period of time. The present art further teaches the need for a long-term rather than short-term treatment for the cessation of leakage and/or re-opening from vessels within the neovasculature being treated while reducing or preventing negative medical outcomes such as loss of visual acuity, retinal damage, et cetera.

Thus, there is a need for an effective method or methods for the treatment of neovasculature disease, in particular neovasculature diseases of the eye, utilizing a PDT treatment which causes the reduction or cessation of leakage and/or re-opening of one or more previously treated or newly occurring vessels within the neovasculature tissue for extended periods of time greater than those currently achieved by convention PDT treatment methodologies.

There is also a need for a PDT treatment method(s) to effectuate closure of leaking, re-opened, and/or newly generated vessels within the neovasculature being treated, which reduces the total number of treatments required by a particular human or animal subject over that of currently available PDT treatment modalities. In doing so, negative outcomes observed from re-treatment via conventional PDT therapies can be minimized or prevented.

As disclosed and claimed herein, the presently described technology addresses one or more of the current problems and disadvantages associated with conventional PDT treatment modalities for neovasculature disease as noted above.

### BRIEF SUMMARY OF THE INVENTION

It has been surprisingly found that the method(s) of the presently described technology disclosed herein can be utilized to treat neovasculature disease, in particular neovasculature disease of an animal or human eye, in a manner in which distinctive and useful properties and outcomes can result.

Further, it has also been surprisingly discovered that the method(s) of the presently described technology reduce or cessate the leakage and/or re-opening of one or more vessels within previously PDT-treated neovasculature tissue, or effectuate closure of any newly generated vessels within that same tissue, for extended periods of time greater than those currently achieved by conventional PDT treatment methodologies.

Moreover, it has been surprisingly found that the method(s) of the presently described technology can minimize or reduce the regular treatment intervals required to effectuate closure of a previously treated or newly generated vessel(s) of the neovasculature tissue. As a result, negative outcomes associated with conventional PDT treatments performed on a subject animal or human on a short-term, but recurrent basis are reduced or prevented.

In at least one aspect, the presently described technology provides a method of treating neovascular disease of the eye by administering at least one vessel occlusive agent to the neovascular tissue of the eye; illuminating the eye with a light having a wave length or waveband that matches the excitation wave length or waveband of the photosensitizing compound to activate the photosensitizing compound to occlude one or more vessels of the neovascular tissue of the eye for a sufficient period of occlusion. Further in this aspect, the vessel occlusive agent can comprise at least one photosensitizing compound that absorbs light in a range of from about 380 nm to about 720 nm while the light utilized has a sufficient light dose, a sufficient pulse duration, and a sufficient duration of illumination that produces a sufficient total fluence of irradiation to achieve occlusion of one or more vessels within the treated neovasculature for an extended period of time.

In another aspect, the present invention provides a method of treating neovascular disease of the eye by administering a sufficient amount of talaporfin sodium sensitizing compound and illuminating the eye with a light having a wave length or waveband that matches the excitation wave length or waveband of the talaporfin sodium photosensitizing compound to activate the compound to occlude one or more vessels of the neovascular tissue of the eye for a sufficient period of occlusion. In this particular aspect, the light utilized has a sufficient light dose, a sufficient pulse duration, and a sufficient duration of illumination that produces a sufficient total fluence of irradiation capable of occluding one or more vessels of the treated neovasculature for an extended period of time.

In a further aspect of the present invention, there is a provided a method of treating neovascular disease of the eye by administering tin ethyl etiopurpurin sensitizing compound and illuminating the eye with a light having a wave length or waveband that matches the excitation wave length or waveband of the photosensitizing compound to activate the photosensitizing compound to occlude one or more vessels of the neovascular tissue of the eye for a period of occlusion of about 15 weeks or greater. For this particular aspect, the light has a sufficient light dose, a sufficient pulse duration, a sufficient duration of illumination that produces a sufficient total fluence of irradiation to photoactivate the tin ethyl etiopurpurin photosensitizing compound to occlude one or more vessels of the treated neovasculature for an extended period of time.

In a still further aspect of the present invention, there is provided a method of treating neovascular disease of the eye by administering verteporfin photosensitizing compound and illuminating the eye with a light having a wave length or waveband that matches the excitation wave length or waveband of the photosensitizing compound to activate the photosensitizing compound to occlude one or more vessels of the neovascular tissue of the eye being treated for a period of occlusion of about 15 weeks or greater. In this particular aspect, the light exhibits a sufficient light dose, sufficient duration of illumination, and sufficient total fluence of irradiation to photoactivate the verteporfin photosensitizing compound to occlude one or more vessels o the treated neovasculature for an extended period of time.

In light of the above, at least one advantage of the presently described technology is a medical care cost savings outcome for the treated subject and healthcare community. Further, enhanced patient care is also believed to be achieved by providing an improved PDT treatment that produces an effect on neovasculature that extends over a longer period of time than currently available.

As compared with currently available PDT treatments, a further advantage of the method(s) of the presently described technology is that such technology utilize may substantially lower cost and utilization of raw materials required to treat a subject base in light of the reduced treatments required to occlude one or more vessels of the vasculature tissue being treated.

### BRIEF DESCRIPTION OF SEVERAL VIEWS OF THE DRAWINGS

FIG. 1 is a table illustrating clinical test results of the presently described PDT treatment method(s).

### DETAILED DESCRIPTION OF THE INVENTION

While the presently described technology will be described in connection with one or more preferred embodiments, it will be understood that it is not limited to those embodiments. On the contrary, the presently described technology includes all alternatives, modifications, and equivalents to those embodiments as may be included within the spirit and scope of the appended claims.

In general, the presently described technology, aspects and embodiments thereof provide methods of treating a subject (human or animal) with a disease that involves neovasculature via an improved PDT treatment that can cause closure (occlusion) of abnormal previously treated and/or newly generated vessels (e.g., blood vessels) for an extended period of time in neovasculature, so that routine re-treatment of the disease is reduced or prevented in comparison to currently available PDT treatment modalities.

In other words, the presently described technology provides one or methods of treating a subject with a disease that involves neovasculature, either as part of the disease manifestation or as blood supply to other diseased tissue, through administration of a photosensitizer and irradiation of the photosensitized tissue to cause closure of previously treated and/or newly generated vessels for an extended period of time, so that re-treatment of the disease is reduced or prevented.

Thus, it should be appreciated by those skilled in the art that the diseases that may be treated with the presently described technology can include, for example, any disease (human or animal) that requires closure of abnormal vessels as part of the therapy utilized. Thus, the present technology can be used to treat a wide range of diseases including, for example, ocular diseases (including, but not limited to (wet) age-related macular degeneration and diabetic retinopathy), oncologic diseases (including, but not limited to those oncologic diseases involving tumors), and diseases of the cardiac and/or vascular systems.

Additionally, and although not wanting to be bound by any particular theory, in at least some embodiments, it is believed that the presently described PDT treatment technology produces an enhanced PDT effect/outcome (i.e., occlusion of previously treated or newly generated vessels in neovasculature tissue for extended period of time) yet, the total light dose utilized may be reduced than the maximum permissible energy input that is medically approved for such treatment.

In accordance with at least one aspect of the presently described technology there is provided a method of treating neovascular disease of the eye, in which a vessel occlusive agent is administered to neovascular tissue; subsequently illuminating the tissue with a light having a wave length or waveband that matches the excitation wave length or waveband of the photosensitizing compound to activate that compound to, in turn, occlude one or more vessels of the neovascular tissue for a sufficient period of occlusion.

It should be appreciated that the vessel occlusive agent of the present technology can be administered directly or indirectly to the neovascular tissue being treated. For example, the vessel occlusive agent can be administered to a subject intravenously, which in turn is capable of delivering the vessel occlusive agent to the neovasculature tissue being treated. Therefore, it is contemplated that any method or apparatus which is capable of introducing and/or delivering the vessel occlusive agent into the subject to be treated, and more particularly introducing or delivering the vessel occlusive agent to the neovasculature to be treated, is within the spirit and scope of the invention as claimed.

With respect to the vessel occlusive agent, the agent includes at least one photosensitizing compound that absorbs light in a range of from about 380 nm to about 720 nm. By way of example only, one or more of the photosensitizing compounds of the presently described technology can absorb light at wavelengths of about 415 nm, about 508 nm, about 664 nm, and about 689 nm, respectively.

Suitable photosensitizing compounds which absorb light in the range of the presently described technology include, but are not limited to porphyrins, purpurins, verteporfin, derivatives thereof, and combinations thereof. Preferably, the photosensitizing compound administered is mono-L-aspartyl-chlorin e6 (also known as talaporfin sodium), LS11 (also known as NPE6), verteporfin, tin ethyl etiopurpurin (also known as SnET2), derivatives thereof, or combinations thereof. Further examples of other photosensitizing compounds that can be used in the practice of the present technology can be found in U.S. Pat. No. 6,800,086 to Strong.

With respect to the light used in the PDT treatment method(s) of the presently described technology, the light preferably exhibits a sufficient light dose, a sufficient pulse duration, and a sufficient duration of illumination that produces a sufficient total fluence of irradiation to activate the photosensitizing compound, which in turn, occludes for an extended period of time (i.e., a sufficient period of occlusion) one or more vessels of the neovasculature being treated.

Additionally, the light source utilized in the performing the method(s) of the presently described technology can be non-coherent light or coherent light. If the light source emits a non-coherent light, then the light source can be, for example, a light emitting diode or ambient light. If the light source emits a coherent light, the light source can be, for example, a laser.

Moreover, typically, but not necessarily, treatment methods of the present described technology may include separate discrete light applications, in series, at one targeted tissue area or multiple targeted tissue areas. Thus, the methods of the present technology can be done as a single procedure (involving a single application of light or a series of light applications), or as a series of procedures (involving a single application of light or a series of light applications).

It will be appreciated by those skilled in the art that any device (e.g., a PDT-based device) that provides light (e.g., laser or non-laser) at the appropriate illumination size and shape, wavelength and irradiance may supply light in accordance with spirit and scope of the presently described technology. For example, 4 separate, immediately successive light applications (in the same treatment session with the subject) of 12 J/cm2 at one targeted tissue site for a total of 48 J/cm2 can be delivered to the targeted neovasculature tissue in the performance of at least one embodiment of the presently described technology.

For example, in at least one embodiment of the presently described technology, the PDT treatment procedure may involve the use of talaporfin sodium as the selected photosensitizer and a laser light dose for an enhanced AMD procedure in which the laser light dose may be in the range of from about 10 to about 50 J/cm2.

The sufficient list dose for the light used to excite the photosensitizing compound, the dose is from about 60 mW/cm² to about 600 mW/cm², more preferably from about 200 mW/cm² to about 300 mW/cm², and most preferably the sufficient light dose is about 300 mW/cm². However, it should be appreciated by those skilled in the art that the light dose used in the practice of the presently described technology can be adjusted based upon the particular photosensitizing compound utilized, the particular neovasculature disease being treated, the particular spot size of the neovasculature tissue being treated, particular patient specific considerations, the particular PDT apparatus utilized, et cetera.

For example, treatment spot size, in general, may be determined based upon the size and shape of the specific targeted tissue area being treated. Typical spot sizes envisaged that can be treated with the improved PDT treatment method(s) of the presently described technology can range from about 500 to about 6000 microns, preferably from about 1200 to about 5500 microns. However, one of ordinary skill in the art will appreciate that the presently described technology can be adjusted to treat spot sizes of further varying size.

Further, those skilled in the art will appreciate that the total light dose utilized to achieve the enhanced occlusive effect of the presently described PDT treatment method(s) can be greater than, up to, or less than total light doses currently approved or mandated as being medically appropriate. Preferably, the total light dose utilized to achieve the enhanced occlusive effect is less than that considered medically appropriate or mandated as the maximum permissible energy input for PDT treatment. Further, it is believed that because extended periods of occlusion can be achieved through use of the PDT treatment method(s) of the presently described technology, repeated use of such methods as a treatment regimen may be reduced. In doing so, it is further believed that negative outcomes associated with PDT treatments such as skin sensitization, photosensitivity and phototoxicity can be substantially reduced or prevented.

Moreover, it is also believed that the present technology reduces a subject's exposure to total light dose and the photosensitizing compound because the parameters/components required for any re-treatment, if required, (e.g., light dose, duration of illumination, amount of photosensitizing compound, et cetera) may be reduced in light of the enhanced occlusive effect initially achieved. In other words, the greater the period of occlusion and extent of that occlusion achieved with the presently described technology will assist in the reduction of further PDT treatments (and parameters thereof) required.

For example, current PDT methods involve the systemic administration of untargeted photosensitive compounds or photosensitizers, the required dosages are relatively high which can lead to skin photosensitivity. The accumulation of photosensitizers in the skin is a property of all systemically administered sensitizers in clinical use. For example, clinically useful porphyrins such as Photophrine® (QLT, Ltd. brand of sodium porfimer) are associated with photosensitivity lasting up to 6 weeks. Purlytin®, which is a purpurin, and Foscan®, a chlorin, sensitize the skin for several weeks. Indeed, efforts have been made to develop photoprotectants to reduce skin photosensitivity (see: Dillon et al., Photochemistry and Photobiology, 48(2):235-238, 1988; and Sigdestad et al., British J. of Cancer, 74:S89-S92, 1996). Typically, PDT protocols involving systemic administration of photosensitizer require that the patient avoid sunlight and bright indoor light to reduce the chance of skin phototoxic reactions.

Thus, it would be beneficial for PDT treatment methods to minimize or reduce the exposure of a treated subject to the photosensitizing agent (e.g., in terms of dose, repeated doses, et cetera), the light source (in terms of amounts of light dose, duration of illumination, repeated doses of light, et cetera), and repeated courses of the treatment regimen, itself. Although not wanting to be bound by any particular theory, it is believed that the PDT method(s) of the presently described technology minimize or reduce exposure of the photosensitizing agent and light source in the treatment of neovasculature disease due to the extended period of occlusion which can be achieved unlike that of conventional PDT treatments.

Sufficient pulse duration for the light presently described technology can from about 30 seconds to about 60 seconds of light per pulse and from about 10 seconds to about 30 seconds between each pulse. More preferably, the sufficient pulse duration is from about 40 seconds of light per pulse and from about 10 seconds between each pulse. Additionally, it should be appreciated by one skilled in the art that the presently described technology contemplates that the pulse duration can be performed one or more times during one or more treatments utilizing the improved PDT method(s).

With respect to the sufficient duration of illumination, the present technology from about 35 seconds to about 220 seconds, more preferably from about 80 seconds to about 120 seconds. However, it should be understood by those skilled in the art that the duration of illumination may be adjusted to achieve a period of illumination necessary to achieve the outcomes and advantages of the presently described technology.

The sufficient total fluence of irradiation of the presently described technology can range from about 30 J/cm² to about 60 J/cm² for a coherent light source. Alternatively, the sufficient total fluence of irradiation can also range from about 40 J/cm² to about 90 J/cm² for an incoherent light source.

Further, the light utilized in performing one or more methods of the present technology should have a sufficient irradiance. Such a sufficient irradiance can range about 50 mW/cm² to about 600 mW/cm² based upon a laser light source. Alternatively, the sufficient irradiance of the present technology can range from about 100 mW/cm² or greater based upon a non-laser light source.

In light of the above, it has been surprisingly found that once the light activates the selected photosensitizing compound(s) of the presently described technology, occlusion within one or more vessels of the neovasculature being treated occurs for extended periods of time. Thus, subjects being treated are able to reduce or minimize the number of PDT treatments required, exposures to the components thereof, and negative outcomes such as treatment side effects (especially those of the photosensitizing compound) due to the extended periods of occlusion achieved. Such extended periods of occlusion are not obtainable via conventional PDT treatments previously known and used.

As a result, the sufficient period of occlusion for the presently described technology can range from about 15 weeks or greater. Preferably, the sufficient period of occlusion can range from about 16 weeks to about 60 months, and more preferably from about 15 weeks to about 6 months depending upon the subject treated and neovasculature disease treated, as well as the one or more photosensitizing compounds and light selected based upon the method(s) of the presently described technology.

In another aspect of the present technology there is provided a method of treating neovascular disease, in particular neovascular disease of the eye, by administering a sufficient amount of talaporfin sodium photosensitizing compound; and illuminating the eye with a light having a wave length or waveband that matches the excitation wave length or waveband of the photosensitizing compound to activate the photosensitizing compound to occlude one or more vessels of the neovascular tissue of the eye for a sufficient period of occlusion. Preferably, the light has a sufficient light dose, a sufficient pulse duration, and a sufficient duration of illumination that produces a sufficient total fluence of irradiation for achieving the period of occlusion desired.

It has been surprisingly discovered that when a photosensitizer such as talaporfin sodium, also known as mono-L-aspartyl-chlorin e6, is used in a PDT method of the present technology, it effectively closes newly generated vessels and maintains new vessel closure for an extended period of time. The closure of such vessels has beneficial effects that depend upon the specific disease being treated. For example, in some cases, diseased tissue that is dependent upon blood supply for nutrients via such newly generated vessels is removed.

In other cases, such as in ocular PDT for (wet) AMD, leakage is reduced or eliminated from previous conventionally treated or newly generated vessels with resultant beneficial flattening of the retina in those cases where fluid caused the retina to bulge forward. The present technology is also useful in the treatment of diabetic retinopathy and other ocular diseases that are associated with neovasculature and/or fluid leakage. As a result, talaporfin sodium is one of the preferred photosensitizers of the present technology because of its now discovered enhanced capability to maintain closure (potentially permanently) of abnormal vessels for longer periods of time than other photosensitizers and conventional PDT treatments.

Moreover, in the case of ocular PDT, and in particular AMD treatment, some subjects may experience an improvement in visual acuity, while most experience a stabilization of vision and/or a slowing of the previous rate of deterioration of vision. Thus, the enhanced vessel closure effect of the present technology may maintain vision, and decrease the rate of retardation of visual acuity. Without being bound to any particular theory, it is believed such outcomes are the result of the closure of vessels and hence reduction in leakage of fluid between the retinal layers or under the retina. The reduction or elimination of fluid leakage, which caused the original bulging, allows the retina to gradually flatten and return to a more normal flat shape thereby improving visual acuity.

The sufficient amount or dose of the talaporfin sodium is between about 0.1 mg/kg to about 2.0 mg/kg based upon the body weight of the particular subject, human or animal, being treated. Further, the sufficient amount of the light dose is between about 10 J/cm2 to about 50 J/cm2 depending upon the particular light source (coherent or incoherent) used.

It will be appreciated by those skilled in the art that the dose of the talaporfin sodium and the light dose (along with other parameters such as those noted below) may be adjusted depending upon the particular neovascular disease being treated. It will be further appreciated by those skilled in the art that the present technology may be used to treat for example, age-related macular degeneration, diabetic retinopathy, or various neovascular tissues in the retina, choroid, or both.

For example, when the photosensitizer in use is talaporfin sodium, and the disease being treated is age-related macular disease (AMD), drug dose may vary with the patient, but is usually within the range from about 0.1 to about 2.0 mg/kg of body weight. Preferably, the drug dose is in the range of about 0.1 to about 1.0 mg/kg, and most preferably about 0.5 mg/kg or less. Clearly, the dose rate will vary depending upon many factors, and therefore the dose is not generally limited by any considerations other than potential toxicity, patient tolerance and the capability to produce an extended “treatment-free” post-PDT period.

With respect to the sufficient total fluence of irradiation for this particular aspect of the present technology, such total fluence can be between about 30 J/cm² to about 60 J/cm² for a coherent light source and between about 40 J/cm² to about 90 J/cm² for an incoherent light source. The sufficient duration of illumination for the light source selected can be from about 35 seconds to about 220 seconds.

In addition, the sufficient pulse duration for the selected light may between about 30 seconds to about 60 seconds of light per pulse and from between 10 seconds to about 30 seconds between each pulse. However, it will be appreciated by those skilled in the art that the pulse duration can be performed one or more times.

It will also be appreciated that the light may also further exhibit a sufficient irradiance of between about 50 mW/cm² to about 600 mW/cm² based upon a laser light source. More preferably, the sufficient irradiance is between about 200 mW/cm² to about 400 mW/cm² for a laser light source, and most preferably is 300 mW/cm². Alternatively, the sufficient irradiance may between about 100 mW/cm² to about 900 mW/cm² based upon a non-laser light source, preferably between about 300 mW/cm² to about 650 mW/cm², more preferably between about 400 mW/cm² to about 550 mW/cm², and most preferably is 525 mW/cm².

Finally, the sufficient period of occlusion for this particular aspect of the present technology can be from about 15 weeks or greater.

In another aspect, the present technology provides a method of treating neovascular disease, in particular neovascular disease of the eye, by administering a sufficient amount of tin ethyl etiopurpurin photosensitizing compound, and illuminating the eye with a light having a wave length or waveband that matches the excitation wave length or waveband of the photosensitizing compound to activate the photosensitizing compound to occlude one or more vessels of the neovascular tissue of the eye for a period of occlusion of about 15 weeks or greater. Preferably, the light has a sufficient light dose, a sufficient pulse duration, a sufficient duration of illumination that produces a sufficient total fluence of irradiation sufficient to achieve the period of occlusion desired. It is also preferably that the light have a wavelength or waveband of about 664 nm.

The sufficient amount or dose of the tin ethyl etiopurpurin photosensitizing compound of the present technology can be from about 0.25 mg/kg to about 1.25 mg/kg based upon the total weight of the subject being treated (human or animal), with about 0.75 mg/kg being most preferred. However, it should be understood by one of ordinary skill in the art that the amount/dose of the tin ethyl etiopurpurin photosensitizing compound may be adjusted depending upon the particular light dose utilized. For example, as the light dose is increased the amount/dose of the tin ethyl etiopurpurin may be decreased, and vice versa.

The sufficient light dose for this particular aspect of the presently described technology can be between about 10 J/cm2 to about 50 J/cm2 while the sufficient pulse duration can be between about 30 seconds to about 60 seconds of light per pulse.

The sufficient duration of illumination can be between about 35 seconds to about 220 seconds such that the light dose, pulse duration and duration of illumination produces a sufficient total fluence of irradiation of between about 30 J/cm² to about 60 J/cm² for a coherent light source and between about 40 J/cm² to about 90 J/cm² for an incoherent light source.

Further, the light can also exhibit a sufficient irradiance. The sufficient irradiance can be between about 50 mW/cm² to about 600 mW/cm² based upon a laser light source, and between about 100 mW/cm² to about 900 mW/cm² based upon a non-laser light source.

As noted above, this particular aspect can be used to treat a variety of neovascular diseases and neovasculature tissue. Preferably, in at least one embodiment, the PDT method utilizing tin ethyl etiopurpurin is used to treat subfoveal choroidal neovascularization.

In a further aspect of the present technology, there is a provided a method of treating neovascular disease of the eye by administering a sufficient amount of a verteporfin photosensitizing compound and illuminating the eye with a light having a wave length or waveband that matches the excitation wave length or waveband of the photosensitizing compound to activate the photosensitizing compound to occlude one or more vessels of the neovascular tissue of the eye for a period of occlusion of about 15 weeks or greater. Preferably, the light has a sufficient light dose, sufficient duration of illumination, and sufficient total fluence of irradiation.

The sufficient amount/dose of the verteporfin photosensitizing compound is an infusion of the compound of about 4 mg/m2 to about 8 mg/m2 over a period of about 15 minutes, with an infusion rate of about 6 mg/m2 over a period of about 15 minutes being most preferred. However, it should be understood by one of ordinary skill in the art that the amount/dose of the verteporfin photosensitizing compound may be adjusted depending upon the particular light dose utilized. For example, as the light dose is increased the amount/dose of the verteporfin photosensitizing compound may be decreased, and vice versa.

In at least one embodiment, the sufficient light dose is between about 10 J/cm2 to about 50 J/cm2 and the sufficient duration of illumination is between about 35 seconds to about 220 seconds that produces the sufficient total fluence of irradiation of between about 30 J/cm² to about 90 J/cm².

The invention and its advantages will be better understood by reference to the following examples. These examples are provided to describe specific embodiments of the invention and to demonstrate how it works. By providing those specific examples, the inventors do not limit the scope of the invention. It will be understood by those skilled in the art that the full scope of the invention encompasses the subject matter defined by the claims concluding this specification, and any equivalents of the claims.

### EXAMPLE

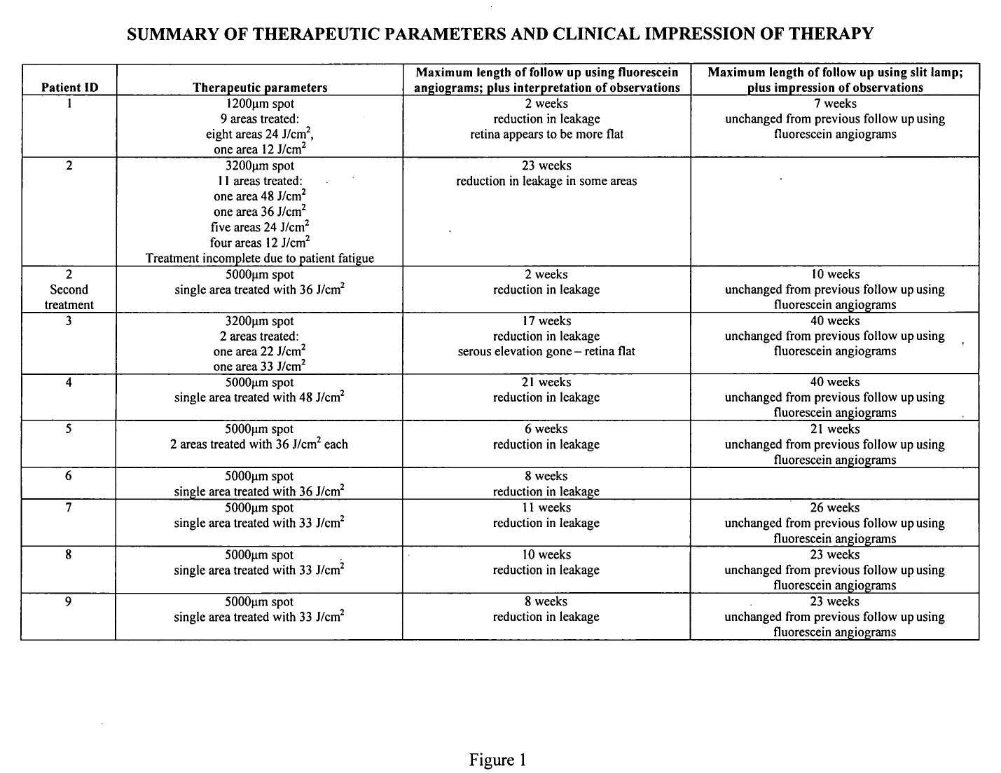

A clinical trial of 9 human subjects with advanced AMD was arranged. The 9 subjects were each treated with PDT using Talaporfin Sodium, and a laser light at 664 nm wavelength as the excitation light source. The Table as provided in FIG. 1 describes the results of and details of the PDT procedures performed on the 9 subjects.

As demonstrated by the results described in FIG. 1, most of the 9 subjects experienced an enhanced neovasculature occlusive effect in the treated areas of the neovasculature for an extended period of time. For example, most patients experienced an enhanced neovasculature occlusive effect for an extended period of about 15 weeks or greater. Yet as noted above, conventional PDT treatment modalities typically achieve an occlusive effect of treated or re-treated neovasculature of about 12 weeks or less, more typically about 4 weeks or less.

Thus, as illustrated from the results in the table of FIG. 1, it is believed that the presently described PDT method(s) achieves a reduction/cessation of leakage, re-leaking, and/or re-opening of previously treated vessels of the neovasculature, abnormal newly generated vessels associated with the closure of the choriocapillaris, closure of the neovasculature supplying the abnormal vessels, and/or closure of the smaller vessels in the choroid.

It is further believed based upon the results of FIG. 1 that the enhanced PDT occlusive effect is not, in essence, “wearing off” and that reduced numbers of vessels are leaking and/or re-opening, requiring re-treatment. As described in FIG. 1, during subsequent observations of the subjects studied there was no indication of re-opening of vessels treated utilizing the PDT treatment of the presently described technology, and as such, no requirement to re-treat the subject. Thus, due to the extended period of time of the occlusive effect, treated subjects required fewer PDT treatments, which in turn reduces negative outcomes, side effects and the like in such subjects.

Additionally, as can be seen in FIG. 1, for subjects 1 and 3, there was leakage that caused serious elevation of the retina. However, after treatment with at least one of the methods of the presently described technology, the retina appeared more flat and leakage had apparently ceased. Such an outcome illustrates the potential benefits of vessel occlusion in neovasculature for extended periods of time. As such, the presently described technology offers benefits and advantages over prior art short-term PDT treatment modalities that cannot reduce or cessate vessel leakage, re-leakage, and/or re-opening for extended periods (e.g., periods of about 15 weeks or greater).

It should be appreciated by those skilled in the art that although talaporfin sodium was used in the illustrative Example, the presently described and claimed technology is not limited to the use of this photosensitizer alone, but rather includes all those PDT treatment methods that meet the selection criteria (i.e., photosensitizer and light characteristics) described herein. Further, while much of the discussion has focused on ocular PDT, especially AMD treatment, the presently described technology is of broader scope and encompasses all PDT procedures where treatment involves the occlusion of abnormal previously treated or newly generated vessels.

The invention is now described in such full, clear, concise and exact terms as to enable any person skilled in the art to which it pertains, to practice the same. It is to be understood that the foregoing describes preferred embodiments of the invention and that modifications may be made therein without departing from the spirit or scope of the invention as set forth herein. Further, the foregoing is an illustrative description of the invention and a person of ordinary skill in the art will appreciate changes and modifications that can be made within the spirit and the scope of the invention as hereinafter claimed.

1. A method of treating neovascular disease of the eye, comprising:
administering at least one vessel occlusive agent to the neovascular tissue of the eye comprising:
at least one photosensitizing compound that absorbs light in a range of from about 380 nm to about 720 nm; and
illuminating the eye with a light having a wave length or waveband that matches the excitation wave length or waveband of the photosensitizing compound to activate the photosensitizing compound to occlude one or more vessels of the neovascular tissue of the eye for a sufficient period of occlusion, and
wherein the light has a sufficient light dose, a sufficient pulse duration, and a sufficient duration of illumination that produces a sufficient total fluence of irradiation.

2. The method of claim 1, wherein the photosensitizing compound administered consists essentially of porphyrins, purpurins, verteporfin, derivatives thereof, and combinations thereof.

3. The method of claim 1, wherein the photosensitizing compound administered is mono-L-aspartyl-chlorin e6.

4. The method of claim 1, wherein the photosensitizing compound administered is verteporfin.

5. The method of claim 1, wherein the photosensitizing compound administered is tin ethyl etiopurpurin.

6. The method of claim 1, wherein the photosensitizing compound administered absorbs light at 664 nm.

7. The method of claim 1, wherein the photosensitizing compound administered absorbs light at 689 nm.

8. The method of claim 1, wherein the photosensitizing compound administered absorbs light at 508 nm.

9. The method of claim 1, wherein the photosensitizing compound administered absorbs light at 415 nm.

10. The method of claim 1, wherein the sufficient period of occlusion is about 15 weeks or greater.

11. The method of claim 10, wherein the sufficient period of occlusion is from about 16 weeks to about 60 months.

12. The method of claim 10, wherein the sufficient period of occlusion is from about 15 weeks to about 6 months.

13. The method of claim 1, wherein the sufficient light dose is from about 60 mW/cm² to about 600 mW/cm².

14. The method of claim 1, wherein the sufficient light dose is from about 200 mW/cm² to about 300 mW/cm².

15. The method of claim 14, wherein the sufficient light dose is about 300 mW/cm².

16. The method of claim 1, wherein the sufficient pulse duration is from about 30 seconds to about 60 seconds of light per pulse and from about 10 seconds to about 30 seconds between each pulse.

17. The method of claim 16, wherein the sufficient pulse duration is about 40 seconds of light per pulse and about 10 seconds between each pulse.

18. The method of claim 16, wherein the pulse duration is performed one or more times.

19. The method of claim 1, wherein the sufficient duration of illumination is from about 35 seconds to about 220 seconds.

20. The method of claim 1, wherein the sufficient duration of illumination is from about 80 seconds to about 120 seconds.

21. The method of claim 1, wherein the light is non-coherent light.

22. The method of claim 21, wherein the non-coherent light is a light emitting diode.

23. The method of claim 22, wherein the non-coherent light is ambient light.

24. The method of claim 1, wherein the light is coherent light.

25. The method of claim 1, wherein the sufficient total fluence of irradiation is from about 30 J/cm² to about 60 J/cm² for a coherent light source.

26. The method of claim 1, wherein the sufficient total fluence of irradiation is from about 40 J/cm² to about 90 J/cm² for an incoherent light source.

27. The method of claim 1, wherein the light further comprises a sufficient irradiance.

28. The method of claim 27, wherein the sufficient irradiance is from about 50 mW/cm² to about 600 mW/cm² based upon a laser light source.

29. The method of claim 27, wherein the sufficient irradiance is about 100 mW/cm² or greater based upon a non-laser light source.

30. The method of claim 1, wherein the neovascular tissue is present in retina, choroid or both.

31. The method of claim 1, wherein the neovascular disease is diabetic retinopathy.

32. The method of claim 1, wherein the neovascular disease is macular degeneration.

33. The method of treating neovascular disease of the eye of claim 1, wherein the method is performed one time or multiple times in the eye.

34. A method of instructing a person to treat neovascular disease of the eye, comprising instructing a person to conduct a method according to claim 1.

35. A method of treating neovascular disease of the eye, comprising:
administering a sufficient amount of talaporfin sodium photosensitizing compound; and
illuminating the eye with a light having a wave length or waveband that matches the excitation wave length or waveband of the photosensitizing compound to activate the photosensitizing compound to occlude one or more vessels of the neovascular tissue of the eye for a sufficient period of occlusion, and
wherein the light has a sufficient light dose, a sufficient pulse duration, and a sufficient duration of illumination that produces a sufficient total fluence of irradiation.

36. The method of claim 35, wherein the sufficient amount of the talaporfin sodium is between about 0.1 mg/kg to about 2.0 mg/kg.

37. The method of claim 35, wherein the sufficient amount of the light dose is between about 10 J/cm2 to about 50 J/cm2.

38. The method of claim 35, wherein the sufficient total fluence of irradiation is between about 30 J/cm² to about 60 J/cm² for a coherent light source and between about 40 J/cm² to about 90 J/cm² for an incoherent light source.

39. The method of claim 35, wherein the sufficient pulse duration is between about 30 seconds to about 60 seconds of light per pulse and from between 10 seconds to about 30 seconds between each pulse.

40. The method of claim 35, wherein the pulse duration is performed one or more times.

41. The method of claim 35, wherein the sufficient duration of illumination is between about 35 seconds to about 220 seconds.

42. The method of claim 35, wherein the light further comprises a sufficient irradiance.

43. The method of claim 42, wherein the sufficient irradiance is between about 50 mW/cm² to about 600 mW/cm² based upon a laser light source.

44. The method of claim 43, wherein the sufficient irradiance is between about 200 mW/cm² to about 400 mW/cm².

45. The method of claim 44, wherein the sufficient irradiance is 300 mW/cm².

46. The method of claim 42, wherein the sufficient irradiance is between about 100 mW/cm² to about 900 mW/cm² based upon a non-laser light source.

47. The method of claim 46, wherein the sufficient irradiance is between about 300 mW/cm² to about 650 mW/cm².

48. The method of claim 47, wherein the sufficient irradiance is between about 400 mW/cm² to about 550 mW/cm².

49. The method of claim 48, wherein the sufficient irradiance is 525 mW/cm².

50. The method of claim 35, wherein the neovascular tissue is present in retina, choroid or both.

51. The method of claim 35, wherein the neovascular disease is diabetic retinopathy.

52. The method of claim 35, wherein the neovascular disease is macular degeneration.

53. The method of claim 35, wherein the sufficient period of occlusion is about 15 weeks or greater.

54. A method of treating neovascular disease of the eye, comprising:
administering a sufficient amount of tin ethyl etiopurpurin photosensitizing compound; and
illuminating the eye with a light having a wave length or waveband that matches the excitation wave length or waveband of the photosensitizing compound to activate the photosensitizing compound to occlude one or more vessels of the neovascular tissue of the eye for a period of occlusion of about 15 weeks or greater, and
wherein the light has a sufficient light dose, a sufficient pulse duration, a sufficient duration of illumination that produces a sufficient total fluence of irradiation.

55. The method of claim 54, wherein the sufficient amount of tin ethyl etiopurpurin photosensitizing compound is from about 0.25 mg/kg to about 1.25 mg/kg based upon the total weight of the subject being treated.

56. The method of claim 55, wherein the sufficient amount of tin ethyl etiopurpurin photosensitizing compound is about 0.75 mg/kg based upon the total weight of the subject being treated.

57. The method of treating neovascular disease of the eye of claim 54, wherein the sufficient light dose is between about 10 J/cm2 to about 50 J/cm2, the sufficient pulse duration is between about 30 seconds to about 60 seconds of light per pulse, and the sufficient duration of illumination is between about 35 seconds to about 220 seconds that produces the sufficient total fluence of irradiation of between about 30 J/cm² to about 60 J/cm² for a coherent light source and between about 40 J/cm² to about 90 J/cm² for an incoherent light source.

58. The method of claim 54, wherein the light further comprises a sufficient irradiance.

59. The method of claim 58, wherein the sufficient irradiance is between about 50 mW/cm² to about 600 mW/cm² based upon a laser light source.

60. The method of claim 58, wherein the sufficient irradiance is between about 100 mW/cm² to about 900 mW/cm² based upon a non-laser light source.

61. The method of claim 54, wherein the neovasculature disease of the eye is subfoveal choroidal neovascularization.

62. The method of claim 54, wherein the wave length or waveband of the light is 664 nm.

63. A method of treating neovascular disease of the eye, comprising:
administering a sufficient amount of verteporfin photosensitizing compound; and
illuminating the eye with a light having a wave length or waveband that matches the excitation wave length or waveband of the photosensitizing compound to activate the photosensitizing compound to occlude one or more vessels of the neovascular tissue of the eye for a period of occlusion of about 15 weeks or greater, and
wherein the light has a sufficient light dose, sufficient duration of illumination, and sufficient total fluence of irradiation.

64. The method of claim 63, wherein the sufficient amount of the verteporfin photosensitizing compound is an infusion rate of from about 4 mg/m² to about 8 mg/m² over a period of about 15 minutes.

65. The method of claim 64, wherein the sufficient amount of the verteporfin photosensitizing compound is an infusion rate of about 6 mg/m² over a period of about 15 minutes.

66. The method of claim 63, wherein the sufficient light dose is between about 10 J/cm2 to about 50 J/cm2 and the sufficient duration of illumination is between about 35 seconds to about 220 seconds that produces the sufficient total fluence of irradiation of between about 30 J/cm² to about 90 J/cm².

67. The method of claim 63, wherein the light further comprises a sufficient irradiance.

68. The method of claim 63, wherein the sufficient irradiance is between about 50 mW/cm² to about 900 mW/cm².
